(19) United States
(12) Patent Application Publication
(10) Pub. No.: US 2024/0065717 A1
(43) Pub. Date: Feb. 29, 2024

(54) Cannulation Devices For Endoscopic Retrograde Cholangiopancreatography (ERCP)

(71) Applicant: Boston Scientific Scimed, Inc., Maple Grove, MN (US)

(72) Inventors: Richard Crawford, Galway (IE); Enda Connaughton, Galway (IE); Anthony O'Brien, Galway (IE); Martin Lawrence Fawdry, Galway (IE); Michael Hughes, Galway (IE); Aiden Flanagan, Galway (IE); Claire Guinane, Galway (IE)

(73) Assignee: Boston Scientific Scimed, Inc., Maple Grove, MN (US)

(21) Appl. No.: 18/236,744

(22) Filed: Aug. 22, 2023

Related U.S. Application Data
(60) Provisional application No. 63/400,366, filed on Aug. 23, 2022.

(51) Int. Cl. A61B 17/32; A61B 17/12; A61M 25/00

(52) U.S. Cl. CPC A61B 17/320016; A61B 17/12131; A61M 25/003; A61M 25/0071; A61B 2017/0034

(57) ABSTRACT

A cannulation device is adapted to gain access to the patient's common bile duct. In some instances, the cannulation device includes a first guidewire lumen extending through an elongate shaft and terminating at a first oblique guidewire port, and a second guidewire lumen extending through the elongate shaft and terminating at a second oblique guidewire port. In some instances, the cannulation device includes an inflatable balloon that is inflatable from a collapsed configuration to an expanded configuration in which the inflatable balloon is adapted to occlude the patient's pancreatic duct. In some instances, the cannulation device includes an expandable element that is expandable from a collapsed configuration in which the expandable element is disposed within the guidewire lumen and an extended configuration in which a portion of the expandable element extends distally from a guidewire port.

### CROSS-REFERENCE TO RELATED APPLICATIONS

This application claims the benefit of priority under 35 U.S.C. § 119 of U.S. Provisional Application No. 63/400,366, filed Aug. 23, 2022, the entire disclosure of which is hereby incorporated by reference.

### TECHNICAL FIELD

The present invention pertains to medical devices and methods for making and using medical devices. More particularly, the present invention pertains to cannulation devices for ERCP.

### BACKGROUND

A wide variety of intracorporeal medical devices have been developed for medical use, for example, intravascular use. These devices are manufactured by any one of a variety of different manufacturing methods and may be used according to any one of a variety of methods. Of the known intracorporeal medical devices and methods for making and using the same, each has certain advantages and disadvantages. There is an ongoing need to provide alternative intracorporeal medical devices as well as alternative methods for making and using intracorporeal medical devices.

### BRIEF SUMMARY

The invention provides design, material, manufacturing method, and use alternatives for intracorporeal medical devices. An example may be found in a cannulation device that is adapted to be advanced through an endoscope to a position proximate a patient's duodenum in order to gain access to the patient's common bile duct. The cannulation device includes an elongate shaft extending to an atraumatic distal tip, a first guidewire lumen extending through the elongate shaft, the first guidewire lumen terminating at a first oblique guidewire port, and a second guidewire lumen extending through the elongate shaft, the second guidewire lumen terminating at a second oblique guidewire port.

Alternatively or additionally, the first oblique guidewire port may be adapted to guide a guidewire extending through the first guidewire lumen in a first direction relative to the atraumatic tip.

Alternatively or additionally, the second oblique guidewire port may be adapted to guide a guidewire extending through the second guidewire lumen in a second direction, different from the first direction, relative to the atraumatic tip.

Alternatively or additionally, the atraumatic distal tip may include a tapered outer surface, with the first oblique guidewire port disposed on a first portion of the tapered outer surface and the second oblique guidewire port disposed on a second portion of the tapered outer surface circumferentially spaced from the first portion of the tapered outer surface.

Alternatively or additionally, the first guidewire lumen may be parallel with the second guidewire lumen within a proximal portion of the cannulation device.

Alternatively or additionally, the first guidewire lumen may diverge radially from the second guidewire lumen within a distal portion of the cannulation device.

Alternatively or additionally, the cannulation device may further include a cutting wire extending through the elongate shaft.

Alternatively or additionally, the cannulation device may be adapted to be advanced through an endoscope.

Alternatively or additionally, the cannulation device may be adapted for accessing the patient's common bile duct from a position proximate the patient's ampulla of vater.

Another example may be found in a cannulation device that is adapted to be advanced through an endoscope to a position proximate a patient's duodenum in order to gain access to the patient's common bile duct. The cannulation device includes an elongate shaft extending to an atraumatic distal tip and a guidewire lumen extending through the elongate shaft, the guidewire lumen terminating at a guidewire port disposed within the atraumatic distal tip. An inflatable balloon is disposed relative to the atraumatic distal tip, the inflatable balloon inflatable from a collapsed configuration to an expanded configuration in which the inflatable balloon is adapted to occlude the patient's pancreatic duct. An inflation lumen extends through the elongate shaft and is fluidly coupled with the inflatable balloon.

Alternatively or additionally, the inflatable balloon may be further adapted to, when inflated, push the atraumatic distal tip away from the patient's pancreatic duct and towards the patient's common bile duct, thereby helping to align the guidewire port with the common bile duct.

Alternatively or additionally, the inflatable balloon may be disposed along a side of the atraumatic distal tip.

Alternatively or additionally, the inflatable balloon, when in its collapsed configuration, may form part of the atraumatic distal tip.

Alternatively or additionally, the cannulation device may be adapted for accessing the patient's common bile duct from a position proximate the patient's ampulla of vater.

Another example may be found in a cannulation device that is adapted to be advanced through an endoscope to a position proximate a patient's duodenum in order to gain access to the patient's common bile duct. The cannulation device includes an elongate shaft extending to an atraumatic distal tip and a guidewire lumen extending through the elongate shaft, the guidewire lumen terminating at a guidewire port disposed within the atraumatic distal tip. An expandable element is disposed within the guidewire lumen, the expandable element expandable from a collapsed configuration in which the expandable element is disposed within the guidewire lumen and an extended configuration in which a portion of the expandable element extends distally from the guidewire port, the expandable element adapted to permit a guidewire to extend through an interior of the expandable element.

Alternatively or additionally, the expandable element may be adapted to occlude the patient's pancreatic duct.

Alternatively or additionally, the cannulation device may be adapted for accessing the patient's common bile duct from a position proximate the patient's ampulla of vater.

Alternatively or additionally, the expandable element may include an eversion-type soft robot.

Alternatively or additionally, the expandable element may include an inverted polymeric sheath.

Alternatively or additionally, an end of the expandable element may be secured relative to a distal end of the cannulation device.

The above summary of some embodiments is not intended to describe each disclosed embodiment or every implementation of the present invention. The Figures, and Detailed Description, which follow, more particularly exemplify these embodiments.

### BRIEF DESCRIPTION OF THE DRAWINGS

The invention may be more completely understood in consideration of the following detailed description of various embodiments of the invention in connection with the accompanying drawings, in which:

FIG. 1 is a schematic view of a portion of the anatomy proximate the duodenum and ampulla of vater;

FIG. 2 is a schematic view of a portion of an illustrative cannulation device;

FIG. 3 is a perspective view of a portion of an illustrative cannulation device;

FIG. 4 is a first end view of the portion of the illustrative cannulation device shown in FIG. 3;

FIG. 5 is a second end view of the portion of the illustrative cannulation device shown in FIG. 3;

FIG. 6 is a schematic view of an illustrative cannulation device positioned proximate the ampulla of vater;

FIG. 7 is a schematic view of an illustrative cannulation device positioned proximate the ampulla of vater;

FIG. 8 is a schematic view of an illustrative cannulation device with an inflatable balloon shown in a deflated configuration;

FIG. 9 is a schematic view of the illustrative cannulation device of FIG. 8 with the inflatable balloon shown in an inflated configuration;

FIG. 10 is a side view of an illustrative cannulation device including an inflatable balloon shown in a deflated configuration;

FIG. 11 is a side view of the illustrative cannulation device of FIG. 10, with the inflatable balloon shown in an inflated configuration;

FIG. 12 is a schematic view of the illustrative cannulation device of FIG. 10, shown in position within the anatomy with the inflatable balloon in the deflated configuration;

FIG. 13 is a schematic view of the illustrative cannulation device of FIG. 10, shown in position within the anatomy with the inflatable balloon in the inflated configuration;

FIG. 14 is a schematic view of an illustrative cannulation device with an expandable element shown in a collapsed configuration;

FIG. 15 is a schematic view of a portion of the illustrative cannulation device of FIG. 14;

FIG. 16 is a schematic view of the illustrative cannulation device of FIG. 14, with the expandable element shown in an extended configuration;

FIG. 17 is a perspective view of a portion of an illustrative cannulation device;

FIG. 18 is a schematic view of the illustrative cannulation device of FIG. 17, shown in position within the anatomy with the expandable element in the collapsed configuration; and

FIG. 19 is a schematic view of the illustrative cannulation device of FIG. 17, shown in position within the anatomy with the expandable element in the extended configuration.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The drawings, which are not necessarily to scale, depict illustrative embodiments and are not intended to limit the scope of the invention.

ERCP (Endoscopic Retrograde Cholangiopancreatography) is a procedure that utilizes both endoscopic and fluoroscopic techniques to diagnose and treat issues arising in the Common bile duct and Pancreatic ducts. To view the ducts endoscopically and fluoroscopically, access through the ampulla of vater is required, and proper position of the papilla is a key in increasing cannulation success. There is a desire to limit or even prevent multiple cannulations of the pancreatic duct, as multiple cannulations of the pancreatic duct can lead to pancreatitis. In some cases, anatomical features, inflammation and adenomas of the papilla or periampullary diverticulum can further complicate attempts to successfully cannulate the common bile duct without irritating the pancreatic duct.

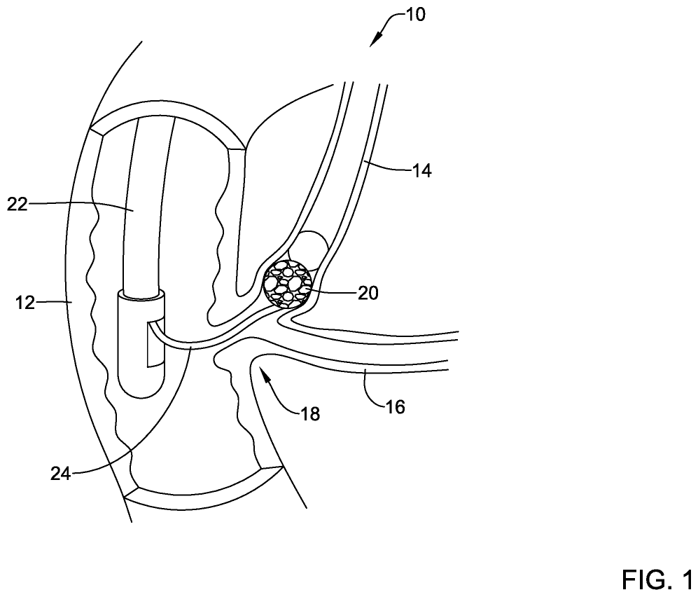

FIG. 1 is a schematic view of a generalized anatomy 10. As shown, the anatomy 10 includes a portion of a duodenum 12. The common bile duct (CBD) 14 and the pancreatic duct (PD) 16 joins the duodenum 12 at the ampulla of vater 18. As shown, one or more gallstones 20 are disposed within the CBD 14. In order to access the CBD 14 in order to remove or break apart the one or more gallstones 20, ERCP (endoscopic retrograde cholangiopancreatography) may be performed. Performing ERCP includes gaining access to the CBD 14 through the ampulla of vater 18. As seen in FIG. 1, this can mean traversing a substantial angle relative to a position within the duodenum 12. Because in part of this substantial angle, it can be difficult to access the CBD 14 instead of accessing the PD 16. As noted, repeatedly cannulating the PD 16 can lead to potential complications.

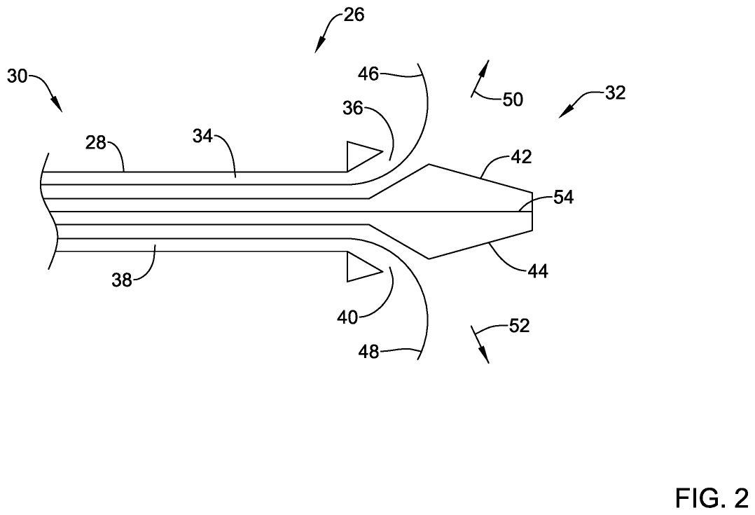

An endoscope 22 is shown positioned within the duodenum 12. A guidewire 24 is shown exiting the endoscope 22, passing through the ampulla of vater 18 and into the CBD 14. FIGS. 2 through 20 provide examples of cannulation devices that may be advanced through the endoscope 22 and used to help guide the guidewire 24 appropriately into the CBD 14 without repeated cannulations of the PD 16, as well as examples of the cannulation devices in use.

FIG. 2 is a schematic view of an illustrative cannulation device 26. The illustrative cannulation device 26 includes an elongate shaft 28 extending from a proximal portion 30 of the cannulation device 26 to a distal portion 32 of the cannulation device 26. In some cases, the cannulation device 26 includes a first guidewire lumen 34 that extends through the elongate shaft 28 and terminates at a first oblique guidewire port 36. A second guidewire lumen 38 extends through the elongate shaft 28 and terminates at a second oblique guidewire port 40. The cannulation device 26 includes an atraumatic distal tip 42. In some cases, the first oblique guidewire port 36 may be considered as being oblique because the first guidewire port 36 is neither parallel to nor at a right angle relative to the first guidewire lumen 34, for example. The second oblique guidewire port 40 may be considered as being oblique because the second guidewire port 40 is neither parallel to nor at a right angle relative to the second guidewire lumen 38. In some cases, the relative angle of the first oblique guidewire port 36 and the second oblique guidewire port 40 may be determined at least in part by an angle at which the tapered outer surface 44 extends.

In some cases, the first oblique guidewire port 36 and the second oblique guidewire port 40 are both disposed within the atraumatic distal tip 42. The atraumatic distal tip 42 may be considered as having a tapered outer surface 44, with the first oblique guidewire port 36 disposed on a first portion of the tapered outer surface 44 and the second oblique guidewire port 40 disposed on a second portion of the tapered outer surface 44 that is circumferentially spaced from the first portion of the tapered outer surface 44. In some cases, at least part of the atraumatic distal tip 42 may be considered as having a frustoconical shape or even a conical shape.

A first guidewire 46 is shown extending through the first guidewire lumen 34 and out the first oblique guidewire port 36 and a second guidewire 48 is shown extending through the second guidewire lumen 38 and out the second oblique guidewire port 40. As can be seen, the first guidewire 46 extends out of the cannulation device 26 in a first direction indicated by an arrow 50 while the second guidewire 48 extends out of the cannulation device 26 in a second direction indicated by an arrow 52. It will be appreciated that depending on the particular orientation of the cannulation device 26 within the duodenum 12, one of the first oblique guidewire port 36 and the second oblique guidewire port 40 may be more closely aligned with the CBD 14 while the other of the first oblique guidewire port 36 and the second oblique guidewire port 40 may be more closely aligned with the PD 16.

In use, a physician or other professional will advance a guidewire through one of the guidewire lumens 34 and 38. As an example, let's assume that the physician or other professional advances the first guidewire 46 through the first guidewire lumen 34 and out the first oblique guidewire port 36. While viewing under fluoroscopy, the physician or other professional will be able to see whether the first guidewire 46 has cannulated the CBD 14 or the PD 16. If they see that the first guidewire 46 has successfully cannulated the CBD 14, the second guidewire 48 will not be used, and the physician or other professional can then continue with the ERCP procedure.

However, if the physician or other professional determines that the first guidewire 46 has cannulated the PD 16, they will leave the first guidewire 46 in place for the time being while they advance the second guidewire 48 through the second guidewire lumen 38 and out the second oblique guidewire port 40. Because the first guidewire 46 is positioned within the PD 16, that means that the second oblique guidewire port 40 may be more closely aligned with the CBD 14. Moreover, having the first guidewire 46 positioned within the PD 16 means that it would be much more difficult to accidently advance the second guidewire 48 into the PD 16 while the first guidewire 46 is already there. Accordingly, advancing the second guidewire 48 through the second guidewire lumen 38 and out the second oblique guidewire port 40 should allow successful cannulation of the CBD 14. Once the CBD 14 has been successfully cannulated, the first guidewire 48 may be withdrawn proximally from the PD 16 and the physician or other professional can then continue with the ERCP procedure. In some cases, the cannulation device 26 may be formed of any of a variety of polymers, such as but not limited to nylon, HDPE (high density polyethylene), PEBAX, Arnitel, Vestamid, Grilamid or a combination of polymers. In some cases, the cannulation device 26 may be a cutting or cauterization wire 54. Wire components may include SS316LVM, Nitinol, platinum or titanium, for example.

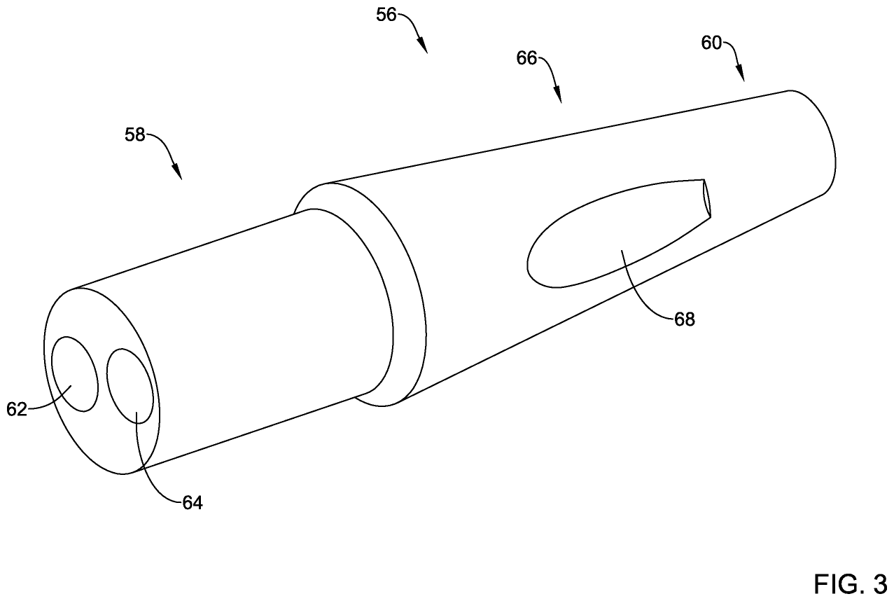

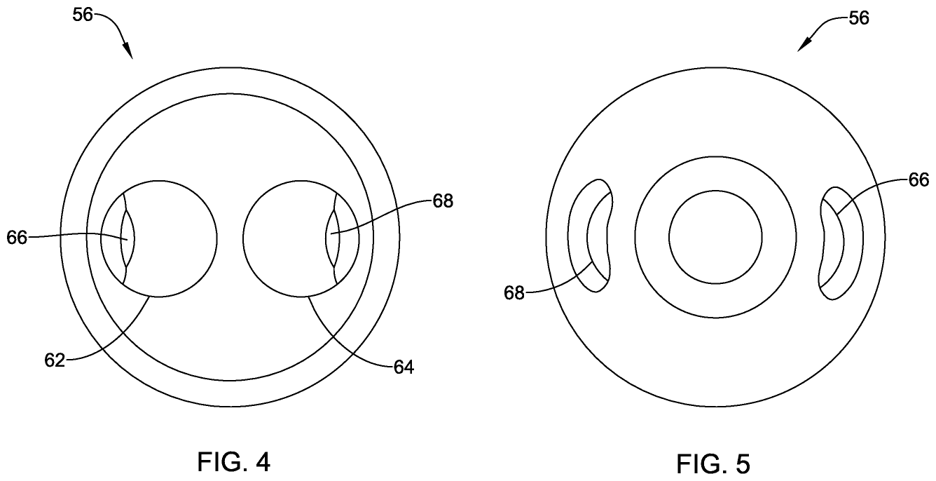

FIG. 3 is a perspective view of a distal portion of an illustrative cannulation device 56 that may be considered as being an example of the cannulation device 26. FIG. 4 is a first end view of the cannulation device 56, showing a proximal end thereof, and FIG. 5 is a second end view of the cannulation device 56, showing a distal end thereof. The cannulation device 56 includes an elongate shaft 58 extending to an atraumatic tip 60. The elongate shaft 58 includes a first guidewire lumen 62 and a second guidewire lumen 64. The first guidewire lumen 62 terminates in a first oblique guidewire port 66 and the second guidewire lumen 64 terminates in a second oblique guidewire port 68. In some cases, the first guidewire lumen 62 and the second guidewire lumen 64 may be considered as being parallel to each other while extending through the elongate shaft 58, and the first guidewire lumen 62 and the second guidewire lumen 64 may radially diverge from each other within the atraumatic tip 60. In some cases, the atraumatic tip 60 may be considered as being disposed at a distal end of the cannulation device 56, with the elongate shaft 58 extending proximally therefrom. In some cases, the cannulation device 56 may be formed of any of a variety of polymers, such as but not limited to nylon, HDPE (high density polyethylene), PEBAX, Arnitel, Vestamid, Grilamid or a combination of polymers. Wire components may include SS316LVM, Nitinol, platinum or titanium, for example.

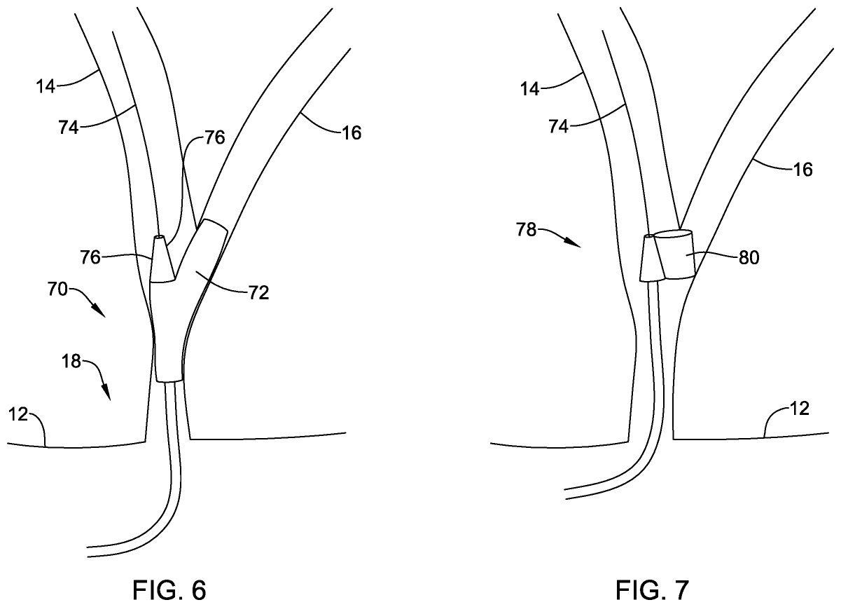

In some cases, instead of being configured to accommodate two guidewires extending through two distinct guidewire lumens, a cannulation device may instead employ an inflatable balloon to increase the likelihood that a single guidewire advanced through the cannulation device will successfully cannulate the CBD 14 rather than the PD 16. FIGS. 6 through 13 provide examples of cannulation devices employing an inflatable balloon.

FIG. 6 is a schematic view of an illustrative cannulation device 70 shown positioned within the ampulla of vater 18. The cannulation device 70 includes an inflatable balloon 72 (shown in an inflated configuration) that is positioned to close off access to the PD 16. As a result, a guidewire 74 exiting an atraumatic tip 76 is able to easily cannulate the CBD 14. Avoiding cannulating the PD 16, and especially avoiding multiple cannulations of the PD 16 while attempting to cannulate the CBD 14, provides improved results. In some cases, the cannulation device 70 may be formed of any of a variety of polymers, such as but not limited to nylon, HDPE (high density polyethylene), PEBAX, Arnitel, Vestamid, Grilamid or a combination of polymers. Wire components may include SS316LVM, Nitinol, platinum or titanium, for example.

FIG. 7 is a schematic view of an illustrative cannulation device 78 shown positioned within the ampulla of vater 18. The cannulation device 78 includes an inflatable balloon 80 (shown in an inflated configuration) that is positioned to close off access to the PD 16. The inflatable balloon 80 is adapted not to extend within the PD 16 (as the inflatable balloon 72 does), but merely to block access to the PD 16. As a result, the guidewire 74 exiting the cannulation device 78 is able to easily cannulate the CBD 14. Avoiding cannulating the PD 16, and especially avoiding multiple cannulations of the PD 16 while attempting to cannulate the CBD 14, provides improved results. In some cases, the cannulation device 78 may be formed of any of a variety of polymers, such as but not limited to nylon, HDPE (high density polyethylene), PEBAX, Arnitel, Vestamid, Grilamid or a combination of polymers. The inflatable balloon 80 may be formed of any medical grade elastomer, such as but not limited to silicone or a thermoplastic elastomer. Wire components may include SS316LVM, Nitinol, platinum or titanium, for example.

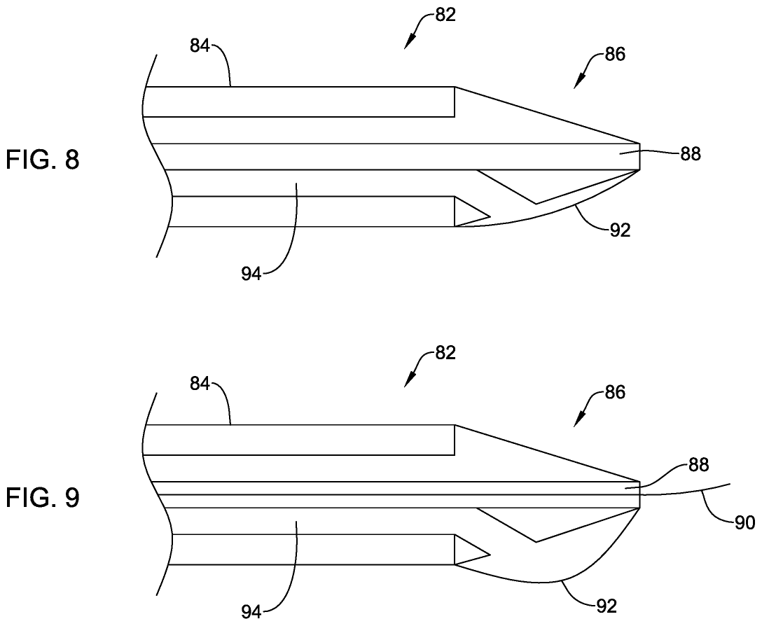

FIG. 8 is a schematic view of an illustrative cannulation device 82 having an inflatable balloon shown in a deflated configuration while FIG. 9 is a schematic view of the illustrative cannulation device 82 with the inflatable balloon shown in an inflated configuration. The cannulation device 82 includes an elongate shaft 84 that extends distally to an atraumatic distal tip 86. A guidewire lumen 88 extends through the elongate shaft 84 and through the atraumatic distal tip. In some cases, as shown in FIG. 9, a guidewire 90 may be extended through the guidewire lumen 88. The cannulation device 82 includes an inflatable balloon 92 and an inflation lumen 94 that extends through the elongate shaft 84 and is fluidly coupled with an interior of the inflatable balloon 92.

In use, a physician or other professional will move the cannulation device 82 into position, and will advance the guidewire 90 through the guidewire lumen 88. While viewing under fluoroscopy, the physician or other professional will be able to see whether the guidewire 90 has cannulated the CBD 14 or the PD 16. If they see that the guidewire 90 has successfully cannulated the CBD 14, the physician or other professional can then continue with the ERCP procedure.

However, if the physician or other professional determines that they have instead cannulated the PD 16, they will withdraw the guidewire 90 and will inflate the inflatable balloon 82. Because the inflatable balloon 82 (when inflated) blocks access to the PD 16, the physician or other professional can once again advance the guidewire 90, which will successfully cannulate the CBD 14. In some cases, the physician or other professional may instead begin the procedure by inflating the inflatable balloon 92 in order to block access to the PD 16 and thus protect the PD 16 against cannulation. In some cases, the cannulation device 82 may be formed of any of a variety of polymers, such as but not limited to nylon, polyurethane, HDPE (high density polyethylene), PEBAX, Arnitel, Vestamid, Grilamid or a combination of polymers. The inflatable balloon 82 may be formed of any medical grade elastomer, such as but not limited to silicone or a thermoplastic elastomer.

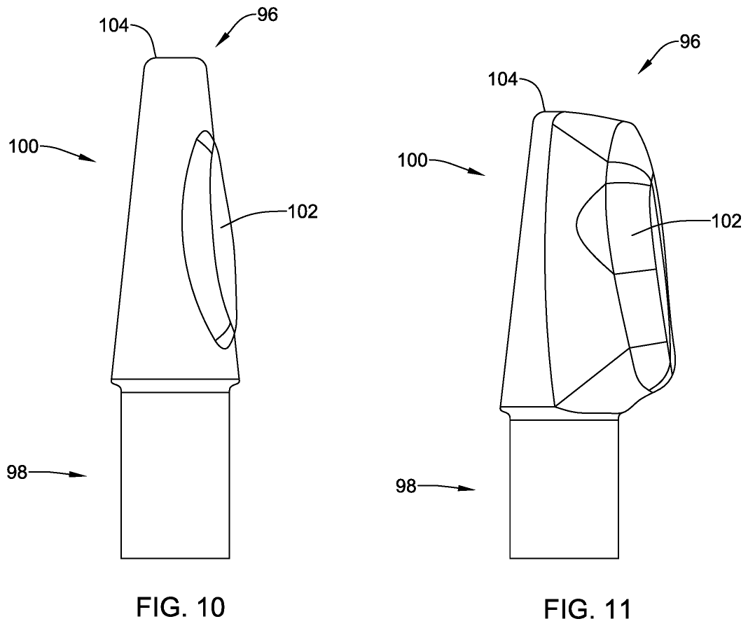

FIG. 10 is a side view of an illustrative cannulation device 96 that may be considered as being an example of the cannulation device 82. The illustrative cannulation device 96 includes an elongate shaft 98 that extends to an atraumatic distal tip 100. The atraumatic distal tip 100 includes an inflatable balloon 102. FIG. 10 shows the inflatable balloon 102 in a deflated configuration. FIG. 11, which is also a side view of the cannulation device 96, shows the inflatable balloon 102 in an inflated configuration. It will be appreciated that when inflated, the inflatable balloon 102 helps to block off access to the PD 16. The inflatable balloon 102, when inflated, also helps to steer a guidewire port 104 away from the PD 16 and towards the CBD 14, particularly when the cannulation device 96 is appropriately positioned.

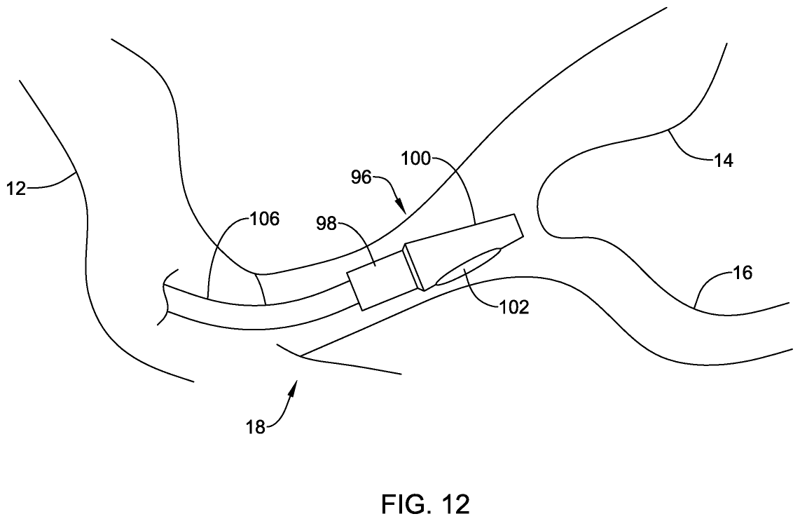

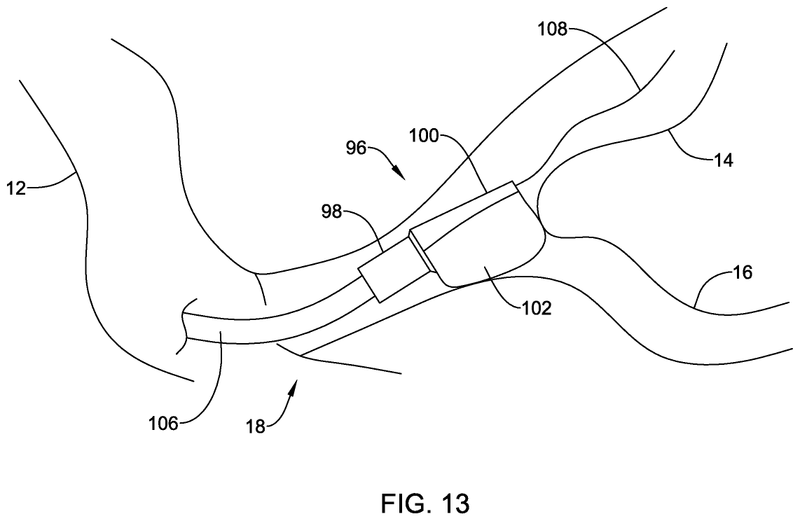

FIG. 12 is a schematic view of the cannulation device 96 positioned within the ampulla of vater 18. As shown, the cannulation device 96 further includes an elongate member 106 extending proximally from the elongate shaft 98. In some cases, the elongate member 106 may be considered as being an extension of the elongate shaft 98, for example. The elongate member 106 may be integrally formed with the elongate shaft 98. FIG. 13 shows the cannulation device 96 positioned within the ampulla of vater 18, but with the inflatable balloon 102 inflated. As a result, the PD 16 is blocked off, and a guidewire 108 extending through the cannulation device 96 is able to successfully reach the CBD 14. In some cases, the cannulation device 96 may be formed of any of a variety of polymers, such as but not limited to nylon, polyurethane, HDPE (high density polyethylene), PEBAX, Arnitel, Vestamid, Grilamid or a combination of polymers.

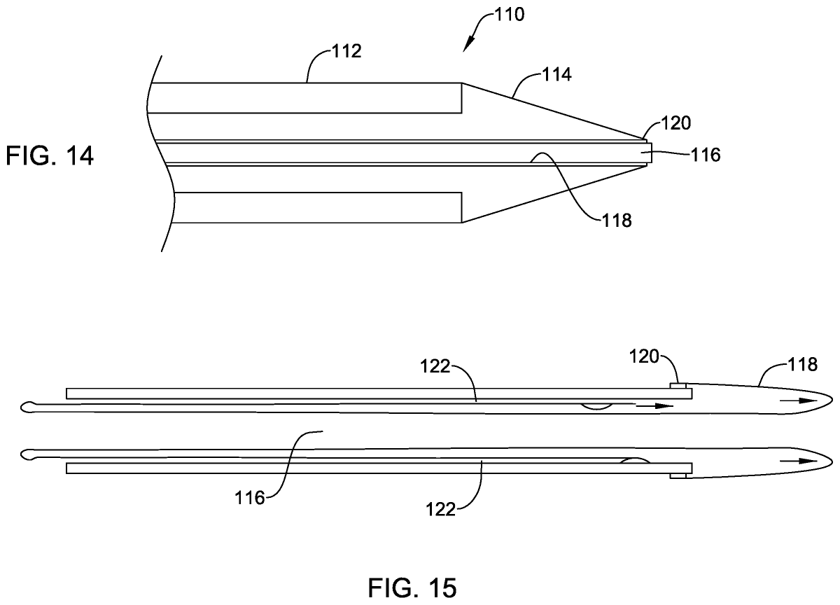

In some cases, a cannulation device may use an expandable element such as but not limited to an eversion-type soft robot in order to increase the likelihood that a single guidewire advanced through the cannulation device will successfully cannulate the CBD 14 rather than the PD 16. FIGS. 14 through 19 provide examples of cannulation devices utilizing an expandable element.

FIGS. 14 and 15 are schematic views of an illustrative cannulation device 110. The illustrative cannulation device 110 includes an elongate shaft 112 that extends to an atraumatic distal tip 114. The cannulation device 110 includes a lumen 116 that extends through the elongate shaft 112 and the atraumatic distal tip 114. The lumen 116 is adapted to accommodate a guidewire (not shown). The lumen 116 is also adapted to accommodate an expandable element 118 that is positioned within the lumen 116. In some cases, the expandable element 118 is itself adapted to accommodate a guidewire extending through the expandable element 118.

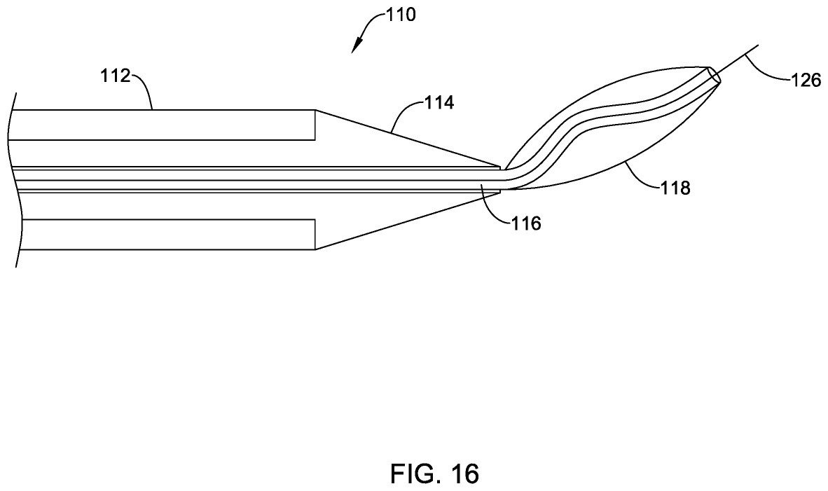

In some cases, the expandable element 118 may be secured relative to the lumen 116 at a point 120. The expandable element 118 may be movable between a collapsed configuration, as seen for example in FIG. 14, and an extended configuration, as partially shown in FIG. 15. In the collapsed configuration, the expandable element 118 extends proximally within the lumen 116 from the attachment point 120. In order to move the expandable element 118 from its collapsed configuration to its extended configuration, a fluid may be advanced through an actuation lumen 122, thereby causing the expandable element 118 to begin everting, and thus extending itself. FIG. 16 shows the cannulation device 110 with the expandable element 118 in its extended configuration. As shown, the expandable element 118 defines a lumen 124 extending through the expandable element 118. The lumen 124 is adapted to accommodate a guidewire 126. In some cases, the cannulation device 110 may be formed of any of a variety of polymers, such as but not limited to nylon, polyurethane, HDPE (high density polyethylene), PEBAX, Arnitel, Vestamid, Grilamid or a combination of polymers. The expandable element 118 may be formed of any medical grade elastomer, such as but not limited to silicone or a thermoplastic elastomer, nylon, Pebax, Grilamid or a combination of suitable polymers.

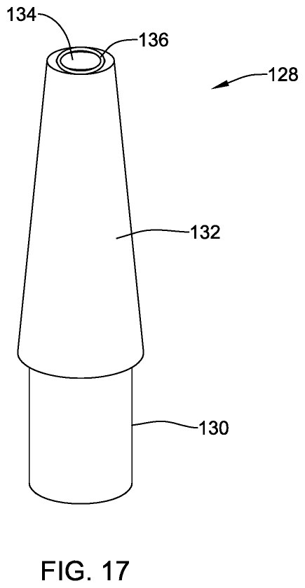

FIG. 17 is a perspective view of an illustrative cannulation device 128 that may be considered as being an example of the cannulation device 110. The cannulation device 128 includes an elongate shaft 130 that extends distally to an atraumatic distal tip 132. A lumen 134 extends through the elongate shaft 130 and the atraumatic distal tip 132, and is adapted to accommodate both a guidewire (not seen in FIG. 17) and an expandable element 136 that is disposed (when in its collapsed configuration) within the lumen 134.

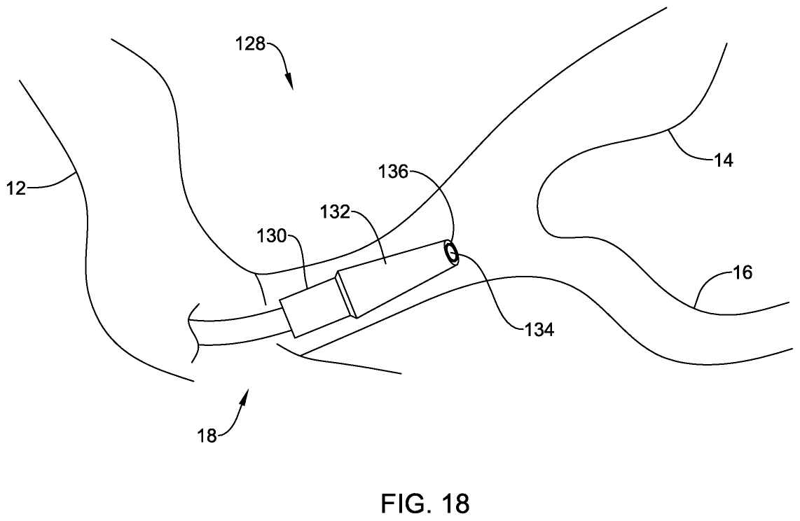

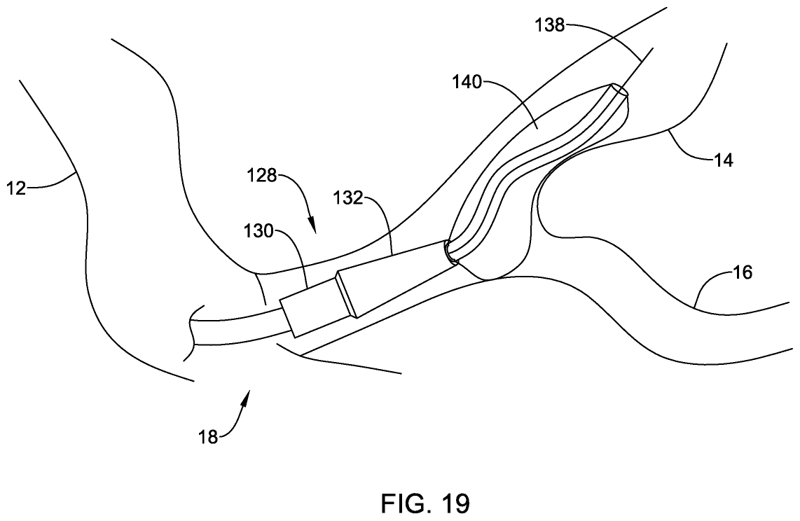

FIGS. 18 and 19 are schematic views of the cannulation device 128 positioned within the ampulla of vater 18. In FIG. 18, the expandable element 136 is shown in its collapsed configuration. In FIG. 19, the expandable element 136 is shown in its extended configuration. In use, a physician or other professional will move the cannulation device 128 into position (as seen for example in FIG. 18), and will advance a guidewire 138 through the lumen 134. While viewing under fluoroscopy, the physician or other professional will be able to see whether the guidewire 138 has cannulated the CBD 14 or the PD 16. If they see that the guidewire 138 successfully cannulated the CBD 14, the physician or other professional can then continue with the ERCP procedure.

However, if the physician or other professional determines that they have instead cannulated the PD 16, they will withdraw the guidewire 138 and will cause the expandable element 136 to expand into its extended configuration in which the expandable element 136 extends into the CBD 14 (as shown in FIG. 19), such as by adding an inflation fluid. They will then extend the guidewire 138 through the cannulation device 128 and through the expandable element 136. Because the expandable element 136 now includes an extended portion 140 that extends directly into the CBD 14, there is no chance of the guidewire 138 missing the CBD 14 and accidently cannulating the PD 16. In some cases, the cannulation device 128 may be formed of any of a variety of polymers, such as but not limited to nylon, polyurethane, HDPE (high density polyethylene), PEBAX, Arnitel, Vestamid, Grilamid or a combination of polymers. The expandable element 136 may be formed of any medical grade elastomer, such as but not limited to silicone or a thermoplastic elastomer, nylon, Pebax, Grilamid or a combination of suitable polymers.

The materials that can be used for the various components of the cannulation devices may include those commonly associated with medical devices. Various components of the cannulation devices described herein may be made from a metal, metal alloy, polymer (some examples of which are disclosed below), a metal-polymer composite, combinations thereof, and the like, or any other suitable material. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

As alluded to above, within the family of commercially available nickel-titanium or nitinol alloys, is a category designated “linear elastic” or “non-super-elastic” which, although may be similar in chemistry to conventional shape memory and super elastic varieties, may exhibit distinct and useful mechanical properties. Linear elastic and/or non-super-elastic nitinol may be distinguished from super elastic nitinol in that the linear elastic and/or non-super-elastic nitinol does not display a substantial “superelastic plateau” or “flag region” in its stress/strain curve like super elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear that the super elastic plateau and/or flag region that may be seen with super elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super elastic nitinol in that linear elastic and/or non-super-elastic nitinol may accept up to about 2-5% strain while remaining substantially elastic (e.g., before plastically deforming) whereas super elastic nitinol may accept up to about 8% strain before plastically deforming. Both of these materials can be distinguished from other linear elastic materials such as stainless steel (that can also can be distinguished based on its composition), which may accept only about 0.2-0.44% strain before plastically deforming.

In some embodiments, the linear elastic and/or non-super-elastic nickel-titanium alloy is an alloy that does not show any martensite/austenite phase changes that are detectable by DSC and DMTA analysis over a large temperature range. For example, in some embodiments, there may be no martensite/austenite phase changes detectable by DSC and DMTA analysis in the range of about −60° C. to about 120° C. in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. In other words, across a broad temperature range, the linear elastic and/or non-super-elastic nickel-titanium alloy maintains its linear elastic and/or non-super-elastic characteristics and/or properties and has essentially no yield point.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Some examples of nickel titanium alloys are disclosed in U.S. Pat. Nos. 5,238,004 and 6,508,803, which are incorporated herein by reference. Other suitable materials may include ULTANIUM™ (available from Neo-Metrics) and GUM METAL™ (available from Toyota). In some other embodiments, a superelastic alloy, for example a superelastic nitinol can be used to achieve desired properties.

In at least some embodiments, various components of the cannulation devices described herein include a radiopaque material including those listed herein or other suitable radiopaque materials.

In some embodiments, a degree of MRI compatibility is imparted into the cannulation devices described herein. For example, to enhance compatibility with Magnetic Resonance Imaging (MRI) machines, it may be desirable to make various components of the cannulation devices described herein, in a manner that would impart a degree of MRI compatibility. For example, various components of the cannulation devices described herein, or portions thereof, may be made of a material that does not substantially distort the image and create substantial artifacts (artifacts are gaps in the image). Certain ferromagnetic materials, for example, may not be suitable because they may create artifacts in an MRI image. Various components of the cannulation devices described herein, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

Some examples of suitable polymers that may be used to form various components of the cannulation devices described herein may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6% LCP.

In some embodiments, the exterior surface of the cannulation devices described herein may include a coating, for example a lubricious, a hydrophilic, a protective, or other type of coating. Hydrophobic coatings such as fluoropolymers provide a dry lubricity which improves device handling and exchanges. Lubricious coatings improve steerability and improve lesion crossing capability. Suitable lubricious polymers may include silicone and the like, polymers such as high-density polyethylene (HDPE), polytetrafluoroethylene (PTFE), polyarylene oxides, polyvinylpyrolidones, polyvinylalcohols, hydroxy alkyl cellulosics, algins, saccharides, caprolactones, and the like, and mixtures and combinations thereof. Hydrophilic polymers may be blended among themselves or with formulated amounts of water insoluble compounds (including some polymers) to yield coatings with suitable lubricity, bonding, and solubility. Some other examples of such coatings and materials and methods used to create such coatings can be found in U.S. Pat. Nos. 6,139,510 and 5,772,609, the entire disclosures of which are incorporated herein by reference.

It should be understood that this disclosure is, in many respects, only illustrative. Changes may be made in details, particularly in matters of shape, size, and arrangement of steps without exceeding the scope of the invention. The invention's scope is, of course, defined in the language in which the appended claims are expressed.

What is claimed is:

1. A cannulation device adapted to be advanced through an endoscope to a position proximate a patient's duodenum in order to gain access to the patient's common bile duct, the cannulation device comprising:
an elongate shaft extending to an atraumatic distal tip;
a first guidewire lumen extending through the elongate shaft, the first guidewire lumen terminating at a first oblique guidewire port; and
a second guidewire lumen extending through the elongate shaft, the second guidewire lumen terminating at a second oblique guidewire port.

2. The cannulation device of claim 1, wherein the first oblique guidewire port is adapted to guide a guidewire extending through the first guidewire lumen in a first direction relative to the atraumatic tip.

3. The cannulation device of claim 2, wherein the second oblique guidewire port is adapted to guide a guidewire extending through the second guidewire lumen in a second direction, different from the first direction, relative to the atraumatic tip.

4. The cannulation device of claim 1, wherein the atraumatic distal tip includes a tapered outer surface, with the first oblique guidewire port disposed on a first portion of the tapered outer surface and the second oblique guidewire port disposed on a second portion of the tapered outer surface circumferentially spaced from the first portion of the tapered outer surface.

5. The cannulation device of claim 1, wherein the first guidewire lumen is parallel with the second guidewire lumen within a proximal portion of the cannulation device.

6. The cannulation device of claim 5, wherein the first guidewire lumen diverges radially from the second guidewire lumen within a distal portion of the cannulation device.

7. The cannulation device of claim 1, further comprising a cutting wire extending through the elongate shaft.

8. The cannulation device of claim 1, wherein the cannulation device is adapted to be advanced through an endoscope.

9. The cannulation device of claim 1, wherein the cannulation device is adapted for accessing the patient's common bile duct from a position proximate the patient's ampulla of vater.

10. A cannulation device adapted to be advanced through an endoscope to a position proximate a patient's duodenum in order to gain access to the patient's common bile duct, the cannulation device comprising:
an elongate shaft extending to an atraumatic distal tip;
a guidewire lumen extending through the elongate shaft, the guidewire lumen terminating at a guidewire port disposed within the atraumatic distal tip;
an inflatable balloon disposed relative to the atraumatic distal tip, the inflatable balloon inflatable from a collapsed configuration to an expanded configuration in which the inflatable balloon is adapted to occlude the patient's pancreatic duct; and
an inflation lumen extending through the elongate shaft and fluidly coupled with the inflatable balloon.

11. The cannulation device of claim 10, wherein the inflatable balloon is further adapted to, when inflated, push the atraumatic distal tip away from the patient's pancreatic duct and towards the patient's common bile duct, thereby helping to align the guidewire port with the common bile duct.

12. The cannulation device of claim 10, wherein the inflatable balloon is disposed along a side of the atraumatic distal tip.

13. The cannulation device of claim 10, wherein the inflatable balloon, when in its collapsed configuration, forms part of the atraumatic distal tip.

14. The cannulation device of claim 10, wherein the cannulation device is adapted for accessing the patient's common bile duct from a position proximate the patient's ampulla of vater.

15. A cannulation device adapted to be advanced through an endoscope to a position proximate a patient's duodenum in order to gain access to the patient's common bile duct, the cannulation device comprising:
an elongate shaft extending to an atraumatic distal tip;
a guidewire lumen extending through the elongate shaft, the guidewire lumen terminating at a guidewire port disposed within the atraumatic distal tip; and
an expandable element disposed within the guidewire lumen, the expandable element expandable from a collapsed configuration in which the expandable element is disposed within the guidewire lumen and an extended configuration in which a portion of the expandable element extends distally from the guidewire port, the expandable element adapted to permit a guidewire to extend through an interior of the expandable element.

16. The cannulation device of claim 15, wherein the expandable element is adapted to occlude the patient's pancreatic duct.

17. The cannulation device of claim 15, wherein the cannulation device is adapted for accessing the patient's common bile duct from a position proximate the patient's ampulla of vater.

18. The cannulation device of claim 15, wherein the expandable element comprises an eversion-type soft robot.

19. The cannulation device of claim 15, wherein the expandable element comprises an inverted polymeric sheath.

20. The cannulation device of claim 15, wherein an end of the expandable element is secured relative to a distal end of the cannulation device.
